(19) United States
(12) Patent Application Publication
(10) Pub. No.: US 2025/0262480 A1
(43) Pub. Date: Aug. 21, 2025

(54) ARRANGEMENT AND PROCESS FOR MONITORING A HUMAN MOBILIZATION PLAN

(71) Applicant: Drägerwerk AG & Co. KGaA, Lübeck (DE)

(72) Inventors: Livio FORNASIERO, Lübeck (DE); Folke WILL, Lübeck (DE); Tim EHLERS, Lübeck (DE)

(21) Appl. No.: 19/055,796

(22) Filed: Feb. 18, 2025

(30) Foreign Application Priority Data
Feb. 19, 2024 (DE) 10 2024 104 516.7

(51) Int. Cl. A63B 24/00; G16H 20/30; G16H 40/67

(52) U.S. Cl. CPC A63B 24/0006; G16H 20/30; G16H 40/67

(57) ABSTRACT

An arrangement and a process monitor a mobilization plan for a human. A movement measuring device includes a holding element which the human wears on the body and measures a movement of the holding element in space and generates a corresponding motion signal. The human also wears a reference element on their body. A radio transmitter, attached to a mobilization aid (an aid for human movement), emits a signal with an identifier of the radio transmitter or the mobilization aid. The mobilization plan specifies a mobilization exercise and a mobilization aid. After selecting the mobilization exercise, a distance between the radio transmitter and the reference element is determined to check whether the mobilization aid specified in the selected mobilization exercise is being used by the human wearing the reference element. Information about the actual performance of the mobilization exercise is determined and compared with the mobilization plan.

### CROSS REFERENCE TO RELATED APPLICATIONS

This application claims the benefit of priority under 35 U.S.C. § 119 of German Application 10 2024 104 516.7, filed Feb. 19, 2024, the entire contents of which are incorporated herein by reference.

### TECHNICAL FIELD

The invention relates to an arrangement and a process for monitoring whether and, if so, how a human performs movements according to a predetermined mobilization plan.

### BACKGROUND

Such a mobilization plan is drawn up, for example, so that a human (human being) performs specific physical movements in accordance with the mobilization plan and thereby improves his/her physical condition. An improvement in physical condition is often necessary after a prolonged period of being bedridden or after obesity, for example. The mobilization plan specifies at least one mobilization exercise. Furthermore, the mobilization plan specifies the frequency and/or the duration with which the human should perform this mobilization exercise for the or each mobilization exercise. As a result of the human carrying out mobilization exercises in accordance with the mobilization plan, their physical condition often improves in many cases.

### SUMMARY

It is an object of the invention to provide an arrangement and a process which support the improvement of a human's physical condition.

The problem is solved by an arrangement with features according to the invention and by a process with features according to the invention. Advantageous embodiments are disclosed. Advantageous embodiments of the arrangement according to the invention are, where appropriate, also advantageous embodiments of the process according to the invention and vice versa.

The arrangement and the process according to the invention are configured to monitor a mobilization plan for a human. This feature means the following: The arrangement and the process are configured to monitor, how human performs physical movements which movements are specified in the mobilization plan period.

The arrangement according to the invention comprises a database. A mobilization plan for a human, for example for a patient, is stored in the database in a computer-evaluable form. The mobilization plan comprises at least one mobilization exercise, preferably several mobilization exercises. On the one hand, the or each mobilization exercise specifies a respective mobilization aid. A mobilization aid is or comprises a device that is configured to mechanically support a human in locomotion and/or to be moved by a human. A wheelchair, a walker, a cane, a walking stick, a stretchable band and a dumbbell are examples of a mobilization aid. On the other hand, the or each mobilization exercise in the mobilization plan specifies a movement that a human should perform with the help of the mobilization aid.

Furthermore, the arrangement comprises at least one mobilization aid radio transmitter. The or each mobilization aid radio transmitter can be attached to a respective mobilization aid. It is possible that the arrangement comprises two mobilization aids and that at least one respective mobilization aid radio transmitter is attached to each mobilization aid. It is possible that at least two mobilization aid radio transmitters are arranged on the same mobilization aid. After attachment, the mobilization aid radio transmitter is ideally not able to perform any relevant movement relative to the mobilization aid, so that the mobilization aid radio transmitter is only moved or can be moved together with the mobilization aid.

The or each mobilization aid radio transmitter is capable of transmitting a signal via radio waves. This signal comprises an identifier of the mobilization aid radio transmitter and/or an identifier of the mobilization aid to which the mobilization aid radio transmitter is currently attached, optionally also a time stamp. This signal can be used to determine from which mobilization aid the signal originates. It is possible that the mobilization aid radio transmitter generates this signal which is transmitted. It is also possible that the radio transmitter receives a signal from a remote sender, modifies the received signal such that the modified signal comprises the identifier and optionally the time stamp, and transmits the modified signal.

The arrangement according to the invention comprises at least one motion measuring device, in one embodiment a plurality of motion measuring devices, and at least one holding element (carrier), in one embodiment a plurality of holding elements. In one implementation, the or each motion measuring device comprises a holding element. In a second implementation, the or each motion measuring device can be mechanically connected to a respective holding element, in one implementation detachably connected. In a third implementation, a data link between the motion measuring device and a spatially remote holding element is established or can be established at least temporarily. In the first and second implementations, the motion measuring device can preferably not move relative to the holding element, but it can in the third implementation.

The or each holding element is configured to be worn by a human on his/her body, in particular on an arm or a leg. The movements of the human, for example those of his/her arm and/or leg, are transferred to the or each holding element that the human wears on their body. Preferably, the holding element does not perform any movement relative to the part of the body on which the holding element is worn. Alternatively, the or each holding element is attached to a mobilization aid or can be attached to a mobilization aid. What a mobilization aid is described further below. It is possible that a first holding element is worn on the body by a human and a second holding element is attached to a mobilization aid.

The motion measuring device can also include a signal processing unit that is spatially removed from the holding element or mechanically connected to the holding element. It is possible that the entire motion measuring device is configured to be worn by a human on his/her body. It is also possible that the motion measuring device is configured as a stationary device or as a mobile device that can be moved without restriction relative to the holding element. It is also possible that the arrangement comprises two motion measuring devices, wherein the first motion measuring device is mechanically connected to the or a holding element and the second motion measuring device can be moved without restriction relative to the holding element.

The or each motion measuring device is configured as follows: The motion measuring device measures a movement in space (spatial motion), wherein the holding element, which is either a part of the motion measuring device or is mechanically connected to the motion measuring device or is data link connected to the motion measuring device, executes this movement. Preferably, the motion measuring device measures the trajectory in space of the holding element. The motion measuring device generates a motion signal. The generated motion signal describes the measured movement of the holding element in space, i.e. it contains information about the measured movement. For example, the motion signal describes the measured trajectory in a specified three-dimensional coordinate system.

The arrangement according to the invention is capable of capturing (recording/acquiring/detecting) an identification (input). This identification identifies a mobilization exercise specified in the mobilization plan, wherein the captured mobilization exercise is to be performed by a patient.

The arrangement according to the invention comprises a reference element and a signal-processing verification unit. The reference element is configured to be worn by a human, for example on the human's arm or leg. When the reference element is worn by a human, the reference element ideally does not move relative to a part of that human's body, with that part of the body wearing (carrying) the reference element. The reference element therefore moves in space together with the body part. It is possible that the or a holding element also functions as a reference element or comprises the reference element. It is also possible that the reference element is carried by the holding element, so that a human carrying the holding element also carries the reference element. It is also possible that the holding element and the reference element are configured to be worn on different parts of a human's body and that the holding element can move relative to the reference element.

As already explained, each mobilization exercise specifies a respective mobilization aid to be used during this mobilization exercise. The arrangement according to the invention is configured as follows: After an identification identifying a mobilization exercise has been captured, the verification (checking) unit automatically checks whether the following situation is actually fulfilled: Is the mobilization aid specified in the mobilization exercise of the captured identification actually used by a human wearing the reference element of the arrangement on his/her body? For this check, the verification unit uses at least a measured distance between the mobilization aid radio transmitter attached to the mobilization aid of the mobilization exercise and the reference element attached to the human body. To measure this distance, the verification unit uses the signal from the mobilization aid radio transmitter and, in particular, the identifier comprised by this signal.

As a rule, the distance between the mobilization aid radio transmitter and the reference element changes when the human moves the mobilization aid. It is therefore preferable to measure the current distance several times. Particularly preferably, the verification unit is able to measure a time course of the distance between the reference element and the mobilization aid radio transmitter. The verification unit is able to use the measured time course to check whether the human wearing the reference element is actually using the mobilization aid.

Preferably, a wireless data link is established or can be established at least temporarily between the or each mobilization aid radio transmitter and the reference element. The verification unit measures the distance at least once, preferably repeatedly, using this data link.

Note: In the following, the formulation is used that a physical quantity is measured. This formulation means that the physical quantity is measured directly or another quantity that correlates with the physical quantity and is therefore an indicator for the physical quantity sought. If the physical quantity being sought is, for example, a distance between a transmitter and a receiver, the indicator used for the distance is, for example, the propagation time or the strength of a signal, wherein the transmitter emits this signal, and the receiver receives this signal and/or reflects it back to the transmitter.

Furthermore, the arrangement according to the invention comprises a signal-processing determination unit. The determination unit is capable of determining information about the or each mobilization exercise that the human actually performs. This means: the determination unit determines whether and, if so, how the human actually performs the mobilization exercise. To determine the information about the performance of a mobilization exercise, the determination unit uses the motion signal and a verification result of the verification unit. As mentioned above, this verification result specifies whether a human actually uses and moves the mobilization aid specified in the mobilization exercise when performing the mobilization exercise. The motion signal describes the movement of the holding element.

Ideally, the human performs the mobilization exercise as specified in the mobilization plan. In practice, there is usually a deviation. A signal-processing comparison unit of the arrangement is configured as follows: The comparison unit compares the information on the actual execution of a mobilization exercise, which the determination unit has determined, with the specification in the mobilization plan for this mobilization exercise. Preferably, the comparison unit causes a result of this comparison to be output in at least one form that can be perceived by a human and/or to be stored in the above-mentioned database or in another database.

The process according to the invention is carried out using such an arrangement. The process according to the invention comprises the corresponding steps. While the process is being performed, the reference element is permanently or at least temporarily connected to the human. The or each mobilization aid radio transmitter is at least temporarily connected to the or a respective mobilization aid. The or each holding element is at least temporarily connected to the human or to a respective mobilization aid while the process is being performed.

The invention therefore provides for the movement of a human to be measured, in particular that of a patient. This human wears the reference element and optionally the holding element and moves the reference element and optionally the holding element. This movement is generated when the human himself/herself moves. Alternatively, the human moves the mobilization aid and thus the holding element on the mobilization aid. The motion signal describes the spatial movement of the holding element. On the other hand, it is checked whether the human is actually using the specified mobilization aid. For this check, the or at least one measured distance between the reference element on the human's body and the mobilization aid radio transmitter on the mobilization aid is used.

The invention makes it possible to automatically check whether a human has performed a specified mobilization exercise with a mobilization aid with sufficient accuracy and has performed the desired movements in space. In particular, the invention makes it possible to automatically check whether the human is actually wearing the reference element and the holding element on his/her body. In particular, the motion signal and the measured distance between the reference element and the mobilization aid radio transmitter can be used for this check.

It is possible, but thanks to the invention not necessary, for a mobilization aid to be attached to the human's body. Thanks to the invention, it is also possible for the human to hold or carry the mobilization aid or move it through the room. Also in this case, it is possible to check whether the human is using the mobilization aid as intended in the mobilization plan.

As a rule, the invention can be used with existing mobilization aids. As a rule, it is sufficient to attach a mobilization aid radio transmitter to an existing mobilization aid. In many cases, the components of the arrangement according to the invention can be realized with the aid of commercially available components.

According to the invention, the distance between the reference element on the human's body and the mobilization aid radio transmitter on the mobilization aid is measured. When the human actually uses the mobilization aid, this measured distance is very small and is usually less than 1 m. Therefore, in many cases, the mobilization aid radio transmitter does not need to emit a strong signal.

In one embodiment, the verification unit is configured as follows: The verification unit decides that a human is using the mobilization aid during a period in which the distance between the reference element on the human's body and the mobilization aid radio transmitter on the mobilization aid is continuously, i.e. during each measurement, less than a predetermined threshold of, for example, 1 m.

The invention can be used in combination with an image recording device (image capture device/imaging device, e.g. a camera) and an image evaluation unit. The image recording device generates a sequence of images, wherein these images show the human while the human is performing the mobilization exercise. By evaluating the images, the image evaluation unit determines whether and, if so, how the human is performing the mobilization exercise. However, the invention eliminates the need to use such an image recording device and such an image evaluation unit. Furthermore, the invention enables but obviates the need for another human, such as a caregiver, to monitor the individual (patient, e.g.) while that individual performs the mobilization exercise. Rather, the invention enables the human to perform the mobilization exercise without being observed by another human or by an image recording device.

In general, the invention eliminates the need to attach relatively large and/or heavy devices to the body of a human performing the mobilization exercise or to a mobilization exercise itself. Thanks to the invention, only the reference element needs to be attached to the body of the human performing the exercise and the or each mobilization aid radio transmitter to a respective mobilization aid being used. In addition, the or each holding element must be attached to the human's body or to a respective mobilization aid. The corresponding devices can be configured to be relatively small and generally consume very little electrical energy.

The invention can be realized with the aid of a stationary infrastructure, for example in a hospital or care home. This stationary infrastructure preferably comprises the database, the or each movement measuring device, the verification unit, the determination unit, and the comparison unit.

In one embodiment, the arrangement comprises a portable device (wearable device). This portable device is configured to be worn by a human on his/her body, in particular on an arm or a leg. Preferably, this human is a patient who is to perform the mobilization exercise. The portable device comprises a receiver. This receiver acts as the reference element or belongs to the reference element. The reference element therefore moves in space together with the portable device and is ideally not capable of performing any movements relative to the portable device, thus maintaining its position relative to the portable device while the portable device is being moved. The verification unit therefore measures the distance between the portable device on the human's body and the mobilization aid radio transmitter on the mobilization aid to be used according to the captured mobilization exercise. The verification unit uses this measured distance as the or a distance between the mobilization aid radio transmitter and the reference element.

Other components of the arrangement according to the invention may belong to this portable device. For example, the verification unit and/or the determination unit are also part of the portable device.

The embodiment with the portable device eliminates the need to provide a stationary infrastructure that records and tracks the movement of the human with the reference element and the holding element as well as the movement in space (spatial motion) of the mobilization aid.

The database and the comparison unit can belong to a stationary computer or to a mobile computer that is physically distant (remote) from a human carrying out the mobilization exercise. For example, the database and the comparison unit are implemented on a smartphone.

According to the invention, the or each motion measuring device is capable of measuring the movement of the associated holding element in space and generating a corresponding motion signal. In one embodiment, the or a motion measuring device comprises a pedometer. The pedometer is connected to the or a holding element mechanically and/or by means of a data link. The pedometer is capable of counting the steps performed by a human while the human is wearing the connected reference element and optionally the connected holding element. The generated motion signal includes information about the measured number of performed steps. This embodiment makes it possible to monitor the performance of a simple mobilization exercise, namely counting the number of steps performed while walking or running. In this embodiment, the or a mobilization aid can be a device that a human uses for walking, for example a cane or a walker or even certain shoes. According to the embodiment, a mobilization aid radio transmitter and optionally a holding element are attached to this device.

The invention is described below by means of embodiment examples. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic overview showing a patient, a caregiver and a mobilization aid;

FIG. 2 is a schematic view showing devices the patient wears on his/her body during the mobilization exercises according to a first embodiment;

FIG. 3 is a schematic view showing a central comparison unit that checks whether the patient has actually performed the mobilization exercise according to the mobilization plan;

FIG. 4 is a schematic view showing devices the patient wears on his/her body during the mobilization exercises according to a second embodiment; and

FIG. 5 is a schematic view showing a central computer system according to the second embodiment.

### DESCRIPTION OF PREFERRED EMBODIMENTS

Referring to the drawings, in one application, the invention is used for the recovery of a patient who has been bedridden for a long time due to intensive medical treatment. At the end of the period of being bedridden, various limbs of the patient are to be trained by means of specific exercises in order to restore the mobility of the limbs. These exercises are known as mobilization exercises. A mobilization aid is used in each mobilization exercise.

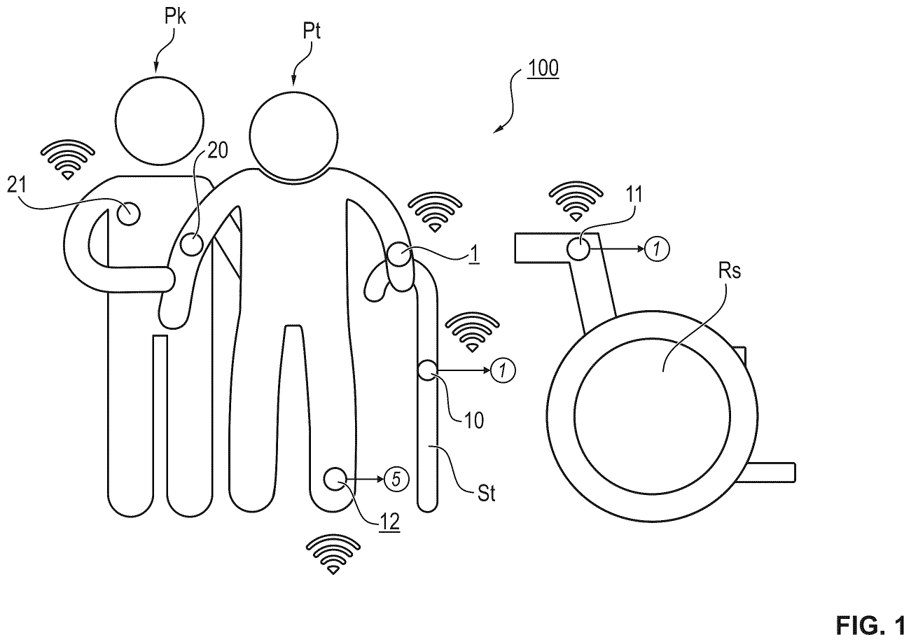

FIG. 1 shows a patient Pt performing exercises to mobilize his/her limbs. In this application, the patient Pt is assisted by a caregiver Pk. In particular, the caregiver Pk prevents the patient Pt from suffering a fall or other accident during the mobilization exercises. The caregiver Pk wears/carries a company ID card (an employee badge/access control card) with a unique identifier. In one embodiment, this ID card comprises a radio transmitter 21.

The mobilization exercises can also be performed without such a caregiver Pk. Whether or not a caregiver is required may depend on the condition of the patient Pt.

Patient Pt performs several mobilization exercises in succession. In each mobilization exercise, patient Pt uses and moves a mobilization aid. A mobilization aid is understood to be a device that mechanically supports the patient Pt in his/her locomotion in space and/or is moved by the patient Pt. By way of example, a first mobilization aid in the form of a wheelchair Rs and a second mobilization aid in the form of a cane St are shown. A mobilization aid may also comprise, for example, a dumbbell or a stretchable band or a pair of shoes.

In a first mobilization exercise, the patient Pt should carry the cane St in one hand, take a predetermined number of steps while supporting himself/herself on the cane St and moving the cane St in the process. Patient Pt does not perform the first mobilization exercise correctly if he/she does not use the cane St at all or does not use it as a walking aid, takes fewer steps with the cane St than specified or does not take the steps with the cane St but, for example, with the help of a walker or while seated.

In a second mobilization exercise, the patient Pt is supposed to move forwards in the wheelchair Rs for a predetermined duration and/or over a predetermined distance and move the wheelchair Rs himself/herself, i.e. turn the two wheels of the wheelchair Rs with his/her arms. Preferably, the patient Pt should move the wheelchair Rs at least once along a curve and/or backwards. The patient Pt does not perform the second mobilization exercise correctly if he/she moves shorter duration or over a shorter distance than specified in the wheelchair Rs or if he/she allows another person to push him/her in the wheelchair Rs or does not use the wheelchair Rs at all.

In a third mobilization exercise, the patient Pt should stand on the spot, hold the cane St in both hands and move the cane St at least over a predetermined distance and for a predetermined duration, for example oscillating up and down and/or to the left or right. Patient Pt does not perform the third mobilization exercise correctly if he/she moves the cane St for too short a distance or for less time than intended or moves his/her arms without the arms supporting the cane St.

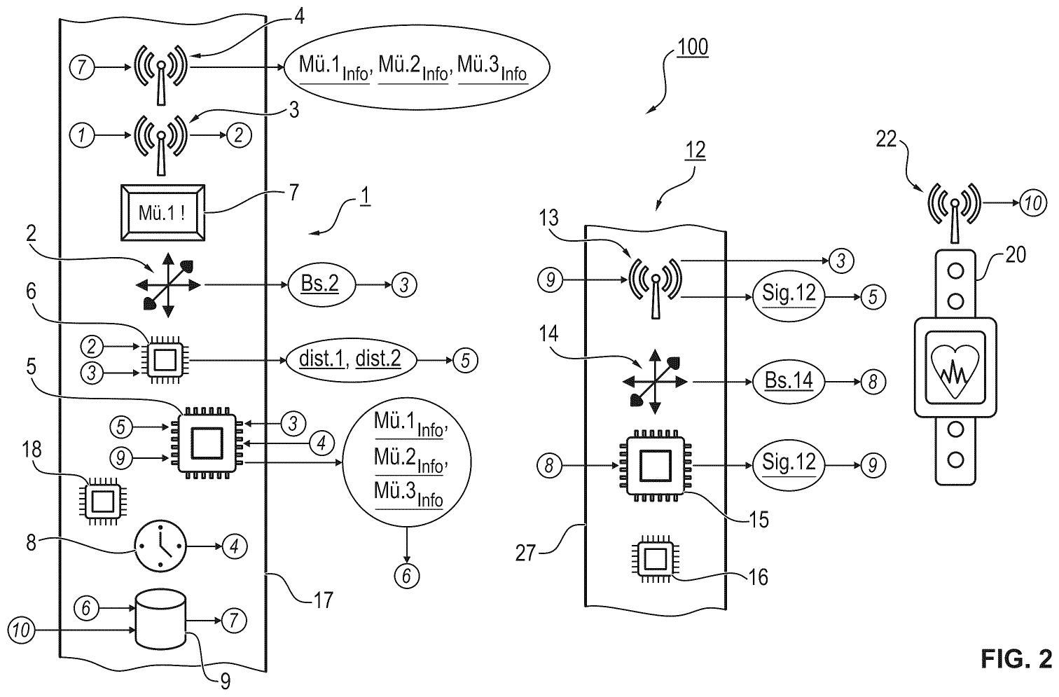

On his/her right arm, patient Pt wears an optional sensor 20 for various vital parameters (vital signs), in particular for his/her current pulse rate, see FIG. 2. The sensor 20 generates a signal that describes the measured vital parameters. A transmitter 22 is able to emit this signal.

A first radio transmitter 10 is attached to the cane St. A second radio transmitter 11 is attached to the wheelchair Rs. Each radio transmitter 10, 11 functions as a mobilization aid radio transmitter and transmits a signal at least during the respective mobilization exercise. This signal comprises a unique identifier of the radio transmitter 10, 11. With the aid of a predetermined computer-evaluable table (data set), it is possible to determine which radio transmitter 10, 11 is currently attached to which mobilization aid Rs, St.

The radio transmitters 10, 11, 21, 22 transmit the respective radio signal using the Bluetooth Low Energy (BLE) or Ultra Wide Band (UWB) transmission protocol, for example. If the BLE transmission protocol is used, the unique identifier is, for example, the MAC address of the radio transmitter 10, 11, 21, 22.

In the embodiment shown in FIG. 1, the patient Pt carries the cane St in his/her left hand and performs the first mobilization exercise while being supported by the caregiver Pk. Patient Pt is wearing a wrist band/bracelet (smart wrist/smart watch) 1 with several components on his/her left arm.

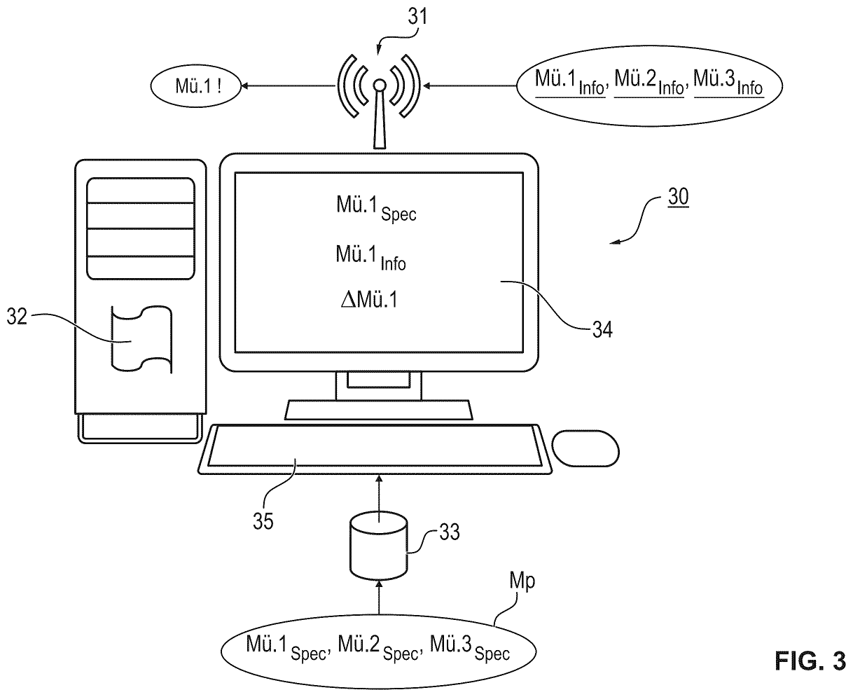

FIG. 2 and FIG. 3 show a first embodiment of the invention. FIG. 2 schematically shows a carrier (holding element) 17 which can be bound around an arm. Furthermore, FIG. 2 schematically shows the following components which are mounted on the carrier 17:
an acceleration sensor 2,
one receiver 3,
a communication unit 4,
a local signal-processing control unit 18,
a signal-processing determination unit 5,
a signal-processing verification unit 6,
an input and output unit 7,
a system clock 8 and
a local database 9.

The same device can implement both the receiver 3 and the communication unit 4.

The receiver 3 acts as the reference element of the embodiment example. The carrier 17 and a carrier 27 described further below each act as a holding element.

In the example shown in FIG. 2, the input and output unit 7 (output MÜ.1 !) indicates that the first mobilization exercise is now to be carried out. Optionally, the mobilization aid St to be used now is displayed, as well as instructions for the use of this mobilization aid St.

A pedometer 12 is attached to the left leg of the patient Pt. The pedometer 12 comprises a carrier 27 which can be tied around a leg. The following further components of the pedometer 12 are mounted on this carrier 27, which are shown schematically in FIG. 2:
an acceleration sensor 14,
a transmitter 13,
a local signal processing unit 15 and
a local control unit 16.

Each acceleration sensor 2, 14 measures its own acceleration in space, preferably both the linear acceleration and the angular acceleration. Preferably, each acceleration sensor 2, 14 is configured as an inertial motion unit.

A motion signal is generated depending on the measured values of the acceleration sensor 2, 14. The motion signal Bs.2, which is generated with measured values from the acceleration sensor 2, describes the movement of the wristband 1 and thus the movement of the patient's left arm Pt in space. The motion signal Bs.14, which is generated with measured values from the acceleration sensor 14, describes the movement of the left leg of patient Pt in space. In one implementation form, the acceleration sensor 2, 14 itself generates the motion signal Bs.2, Bs.14. It is also possible that the respective signal processing unit 5, 15 receives and processes measured values from the acceleration sensor 2, 14 and generates the respective motion signal Bs.2, Bs.14.

The signal processing unit 15 of the pedometer 12 processes the motion signal Bs.14 and generates a signal Sig.12, which describes the number of steps taken by the patient Pt. Preferably, the signal Sig.12 also characterizes the movement path covered by the pedometer 12, for example the horizontal distance or the total distance covered in a three-dimensional space. The control unit 16 causes the transmitter 13 of the pedometer 12 to emit the signal Sig.12.

The receiver 3 is able to receive the respective signal from each radio transmitter 10, 11 and the signal Sig.12 from the transmitter 13 of the pedometer 12 and the signal with the vital parameters from the optional transmitter 22.

The verification unit 6 processes the signals from the radio transmitters 10 and 11. By processing the signal from the radio transmitter 10, the verification unit 6 measures the time course (progression) of the distance dist.1 between the radio transmitter 10 on the cane St and the receiver 3 on the arm of the patient Pt. This distance dist.1 corresponds approximately exactly to the distance between the cane St and the left arm of the patient Pt, who is wearing the cane St and the wristband 1. In addition, the verification unit 6 measures the time course of the distance dist.2 between the radio transmitter 11 on the wheelchair Rs and the receiver 3. Because each signal of a radio transmitter 10, 11 comprises an identifier of this radio transmitter 10, 11, the verification unit 6 is able to determine which measured distance relates to which mobilization aid Rs, St.

In one implementation, the verification unit 6 uses the strength of a signal (Received Signal Strength Indicator) of a radio transmitter 10, 11 to determine the distance between the receiver 3 and the radio transmitter 10 or 11. In another implementation, the propagation time of a signal from the receiver 3 to the radio transmitter 10, 11 and back to the receiver 3 is measured in order to determine the distance.

The verification unit 6 applies a predetermined decision criterion to the measured distance between the radio transmitter 10 on the cane St and the receiver 3. By applying the decision criterion, the verification unit 6 automatically decides whether or not the patient Pt has used the cane St with the radio transmitter 10 during the entire course of the first mobilization exercise and during the entire course of the third mobilization exercise. For example, the verification unit 6 checks whether the distance between the radio transmitter 10 and the receiver 3 is less than or equal to a predetermined first threshold during the entire first mobilization exercise. In addition, the verification unit 6 uses the motion signal Bs.2 to check whether the patient Pt moves the cane St with his/her left arm during the first and third mobilization exercise. Accordingly, the verification unit 6 automatically decides whether the patient Pt is sitting in the wheelchair Rs during the second mobilization exercise.

The determination unit 5 receives the verification result from the verification unit 6. In addition, the determination unit 5 receives the signal Sig.12 from the transmitter 13 of the pedometer 12. As already mentioned, the received signal Sig.12 describes the number of steps and identifies the movement path that the pedometer 12 has covered. Furthermore, the determination unit 5 receives a signal from the system clock 8 and determines the time period in which the patient Pt performed the first, second and third mobilization exercise and used the respective mobilization aid St, Rs.

The control unit 18 causes the determined actual result Mü.1_{Info} of the first mobilization exercise to be stored in the local database 9. In other words, what the patient Pt actually did during the first mobilization exercise is stored. The following information Mü.1_{Info} about the first mobilization exercise is stored in the local database 9:
how many steps the patient has performed Pt,
the movement path that the pedometer 12 has traveled during these steps,
the time period in which the patient Pt performed these steps,
whether the patient Pt used the cane St during this period and
optional values for the patient's vital parameters Pt have been measured.

Optionally, the control unit 18 has the following effect: While the patient Pt is performing the first mobilization exercise, an intermediate result is displayed on the input and output unit 7, for example how many steps the patient Pt has taken so far and the distance he/she has covered in the process. Optionally, an error message is also displayed, for example if the verification unit 6 has determined that the patient Pt has not used the cane St.

In the second mobilization exercise, the patient Pt is to move the wheelchair Rs and cover a certain distance during the movement. The verification unit 6 checks whether the patient Pt is actually sitting in the wheelchair Rs during the second mobilization exercise. By evaluating the motion signal Bs.2, the determination unit 5 determines whether the patient Pt is actually moving the wheelchair Rs with his/her arms, i.e. whether he/she is moving the two wheels of the wheelchair Rs himself/herself, or whether he/she is being pushed by another person. In addition, the approximate distance covered by the wristband 1 during the second mobilization exercise is determined. In addition, the time period in which the second mobilization exercise is performed is determined.

The control unit 18 causes the following information Mü.2_{Info} about the second mobilization exercise to be stored in the database 9:
the distance covered by wristband 1 during the second mobilization exercise,
whether the patient Pt used the wheelchair Rs during the second mobilization exercise and moved it himself/herself,
the period during which patient Pt performed the second mobilization exercise, and
optional vital parameters of the patient Pt measured during the second mobilization exercise.

In the third mobilization exercise, the patient Pt should stand on the spot and move the cane St with both hands. The determination unit 5 determines, on the one hand, whether the patient Pt is actually moving the cane St during the third mobilization exercise and, on the other hand, which movement the wristband 1 and thus the left arm of the patient Pt are performing. The control unit 18 causes corresponding information Mü.3_{Info} about the third mobilization exercise to be stored in the local database 9.

FIG. 3 shows an example of a computer 30. In the example shown, the computer 30 is a stationary device, such as a PC. The computer 30 can also be a portable computer, for example a smartphone.

The computer 30 comprises an output unit 34, an input unit 35 and a communication unit 31. At least temporarily, a data link is established between the communication unit 31 of the computer 30 and the communication unit 4 of the wristband 1 via radio waves.

A mobilization plan Mp for the patient Pt is stored in a database 33 in a form that can be evaluated by a computer. In the embodiment example, the mobilization plan Mp specifies the mobilization exercises just described. For the first mobilization exercise, the mobilization plan Mp specifies how many steps the patient Pt should take during the first mobilization exercise and that he/she should use the cane St for this. For the second mobilization exercise, the mobilization plan Mp specifies the distance that patient Pt should cover in wheelchair Rs and that he/she should move wheelchair Rs himself/herself. For the third mobilization exercise, the mobilization plan Mp specifies how often and over what distance the patient Pt should move the cane St with his/her arms. For each mobilization exercise, the mobilization plan Mp also specifies how often this mobilization exercise should be performed in a given mobilization period. A specification Mü.1_{Spec}, Mü.2_{Spec}, Mü.3_{Spec} is therefore stored for each mobilization exercise, wherein this specification Mü.1_{Spec}, Mü.2_{Spec}, Mü.3_{Spec} comprises the requirements described above.

It is possible that the mobilization plan Mp is created by a human and stored in the database 33. In another form of implementation, the mobilization plan Mp is generated automatically. To generate the mobilization plan Mp automatically, the following data about the patient Pt is used:
Height, weight, age and sex of the patient Pt and
Type of procedure performed, in particular limbs affected.

Applications for automatically generating a mobilization plan are used, for example, under the names
“PhysioMate—Customize Your Patient Sessions” (https://physiomate.be) and
“Mifysio—get more time to help people” (https://mifysio.app).

It is also possible to set up the mobilization plan Mp using a learning procedure (machine learning). The learning process uses a sample with several sample elements. Each sample element comprises patient data for a specific patient on the one hand and a mobilization plan for this patient on the other. The mobilization plan of a sample element was created by a human or was generated automatically and checked by the humans and adjusted if necessary. The application of the learning procedure to the sample provides an initial mobilization plan for patient Pt. A human checks this initial mobilization plan. The result of the checking is the mobilization plan Mp, which is stored in the database 33 and subsequently used.

In the example shown, a user selects a mobilization exercise, for example the first mobilization exercise. The communication unit 31 transmits the specification that the first mobilization exercise is now to be performed to the communication unit 4 of the wristband 1. The input and output unit 7 indicates that the first mobilization exercise is to be performed (output Mü.1 !). It is also possible for the patient Pt himself/herself or the caregiver Pk or someone else to use the input and output unit 7 to indicate that the first mobilization exercise is now to be performed.

A signal-processing comparison unit 32 is implemented on the computer 30, preferably as a software program. The comparison unit 32 has at least temporary read access to the database 33 in which the mobilization plan Mp is stored.

The comparison unit 32 captures the information Mü.1_{Info}, Mü.2_{Info}, Mü.3_{Info} about the three mobilization exercises, which have been stored in the local database 9 and transmitted to the computer 30 with the aid of the communication units 4 and 31. The comparison unit 32 compares this information Mü.1_{Info}, Mü.2_{Info}, Mü.3_{Info} with the respective specification Mü.1_{Spec}, Mü.2_{Spec}, Mü.3_{Spec}, which is stored as part of the mobilization plan Mp in the database 33. The comparison unit 32 determines a deviation between the specification of a mobilization exercise in the mobilization plan Mp and the transmitted information about the actual execution of this mobilization exercise. The result of the comparison is displayed visually on the output unit 34. As an example, FIG. 3 shows that the stored specification Mü.1_{Spec}, the transmitted information Mü.1_{Info} and the determined deviation ΔMü.1 for the first mobilization exercise are output on the output unit 34.

In the example shown, the mobilization plan Mp is stored in a database 33 of a computer 30, wherein the computer 30 is spatially separated from the wristband 1. The comparison unit 32 also belongs to this computer 30. It is also possible that the mobilization plan Mp is stored in the local database 9 of the wristband 1 and the comparison unit 32 is also a component of the wristband 1. In this embodiment, no remote computer 30 and no data communication between the wristband 1 and this computer 30 are required.

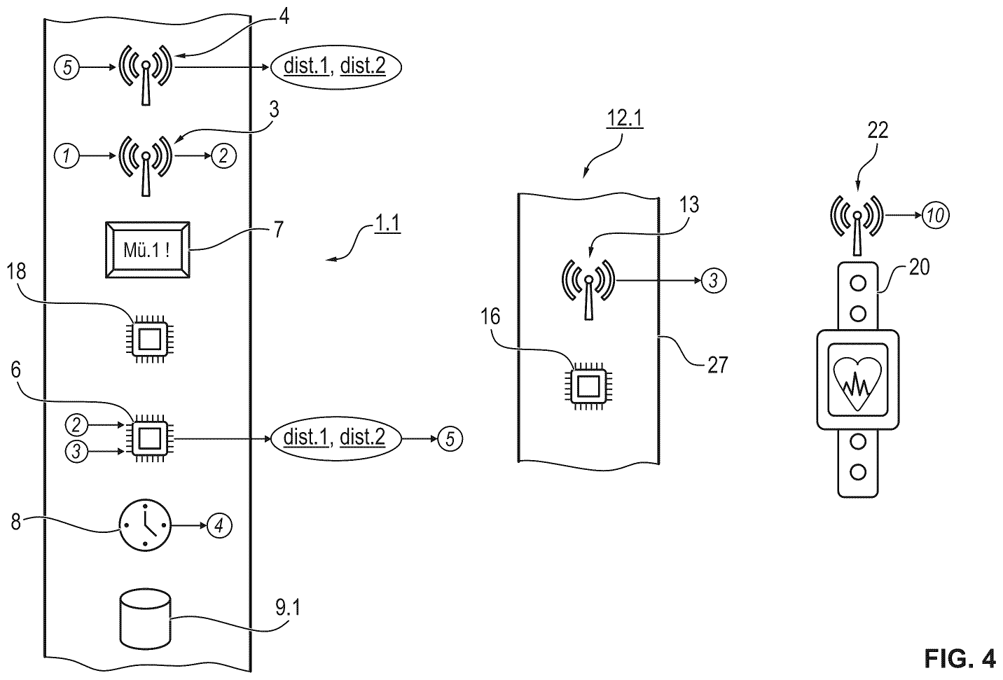

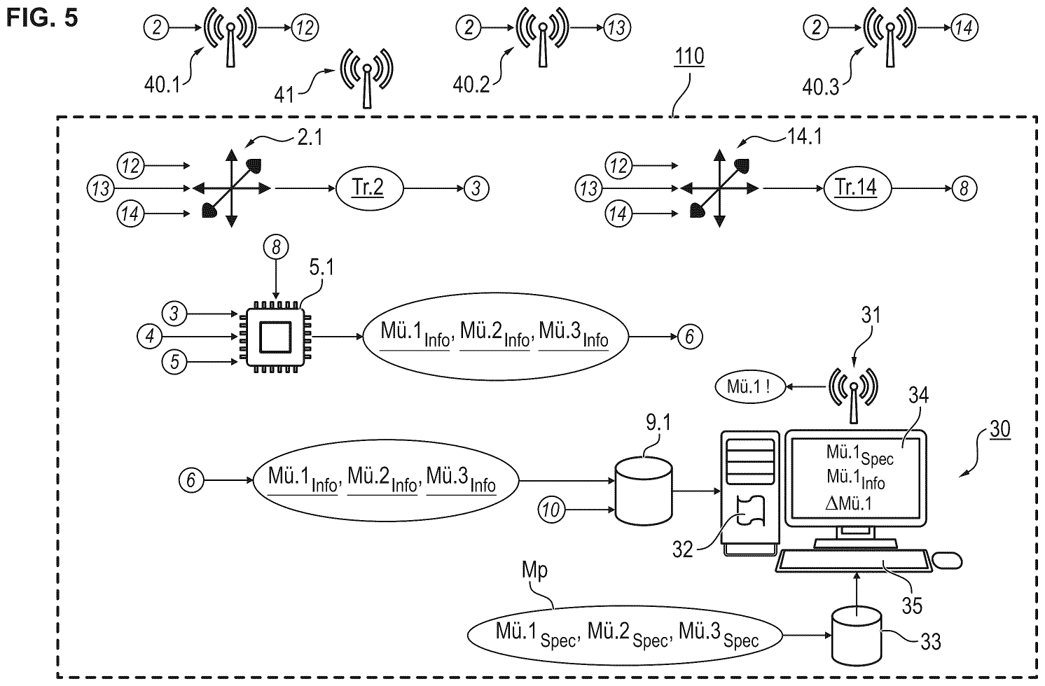

FIG. 4 and FIG. 5 show a second embodiment of the invention. The same reference characters have the same meaning as in the first embodiment according to FIG. 2 and FIG. 3. Instead of the wristband 1, a wristband 1.1 with a reduced range of functions is used in the second embodiment. Instead of the pedometer 12, a pedometer 12.1 with a reduced range of functions is used.

A computer system 110 is spatially separated from the wristband 1.1 and the pedometer 12.1 and can be configured as a stationary device or as a portable computer, for example as a smartphone, see FIG. 5. The computer system 110 comprises the following components:
a tracking unit (locator) 2.1 instead of the acceleration sensor 2 on the carrier 17,
a tracking unit (locator) 14.1 instead of the acceleration sensor 14 on the carrier 27,
a determination unit 5.1 instead of the determination unit 5 on the carrier 17,
a database 9.1 for the information Mü.1_{Info}, Mü.2_{Info}, Mü.3_{Info} instead of the database 9 on the carrier 17,
a communication unit 41,
the computer 30 and
the database 33 with the mobilization plan Mp.

The second implementation is preferably used in a room where an infrastructure is available, e.g. in a hospital. Several fixed receivers 40.1, 40.2, 40.3 are installed in this room. These receivers 40.1, 40.2, 40.3 receive signals from the communication unit 4 of the wristband 1.1 and from the transmitter 13 of the pedometer 12.1. These signals are transmitted by the receivers 40.1, 40.2, 40.3 to the tracking units 2.1 and 14.1 and form motion measuring devices. Using these signals, the tracking units 2.1 and 14.1 generate two trajectories Tr.2 and Tr.14. The trajectory Tr.2 describes the movement of the wristband 1.1 in space, the trajectory Tr.14 describes the movement of the pedometer 12.1 in space. The trajectories Tr.2 and Tr.14 preferably refer to a predetermined three-dimensional stationary coordinate system. With the aid of the communication units 4 and 41, the computer system 110 also receives the time courses of the distances dist.1, dist.2.

The determination unit 5.1 evaluates the trajectories Tr.2 and Tr.14 and the measured time courses of the distances dist.1, dist.2 in order to determine the information about the three mobilization exercises Mü.1_{Info}, Mü.2_{Info}, Mü.3_{Info}. This information Mü.1_{Info}, Mü.2_{Info}, Mü.3_{Info} is stored in the database 9.1.

In the embodiments that have just been described with reference to FIGS. 2 to 5, the verification unit 6 on the wristband 1, 1.1 measures the time course of the distance dist.1, dist.2 between the wristband 1, 1.1 and the cane St and between the wristband 1, 1.1 and the wheelchair Rs. It is also possible that this verification unit 6 is also a component of the computer system 110. A positioning unit of the computer system 110 receives the two signals from the radio transmitters 10 and 11 and determines the respective trajectory of the cane St and the wheelchair Rs in space and derives the distances dist.1, dist.2 from this.

While specific embodiments of the invention have been shown and described in detail to illustrate the application of the principles of the invention, it will be understood that the invention may be embodied otherwise without departing from such principles.

List of reference characters

| | |
|---|---|
| 1 | Wristband (bracelet), comprises the acceleration sensor 2, the receiver 3, |
| | the communication unit 4, the determination unit 5, the verification unit |
| | 6, the input and output unit 7, the system clock 8, the database 9 and the |
| | carrier 17 |
| 1.1 | Wristband with reduced functionality, comprising the receiver 3, the |
| | communication unit 4, the input and output unit 7, the system clock 8 and |
| | the carrier 17 |
| 2 | Acceleration sensor of the wristband 1 |
| 2.1 | Tracking unit, belongs to the computer system 110, determines the |
| | trajectory Tr.2 of the wristband 1.1, using signals from the receivers 40.1, |
| | 40.2, 40.3 |
| 3 | Receiver of the wristband 1, 1.1, receives a signal from each of the radio |
| | transmitters 10, 11, acts as the reference element |
| 4 | Communication unit of the wristband 1, is in a data link with the |
| | communication unit 31 of the computer 30 |
| 5 | Signal-processing determination unit of the wristband 1, generates the |
| | information Mü.1_{Info}, Mü.2_{Info}, Mü.3_{Info}, uses the motion signal Bs.2 and |
| | the signal Sig.12 for this purpose |
| 5.1 | Signal-processing determination unit of the computer system 110 |
| 6 | Signal-processing verification unit of the wristband 1, 1.1, receives |
| | signals from the radio transmitters 10 and 11, determines the time courses |
| | of the distances dist.1 and dist.2 |
| 7 | Input and output unit of the wristband 1, 1.1 |
| 8 | System clock of the wristband 1, 1.1 |
| 9 | Local database of the wristband 1, in which the captured information |
| | Mü.1_{Info}, Mü.2_{Info}, Mü.3_{Info} about the mobilization exercises is stored |
| 9.1 | Local database of the wristband 1.1 |
| 10 | Radio transmitter on the cane St, acts as a mobilization aid radio |
| | transmitter |
| 11 | Radio transmitter on wheelchair Rs, acts as a mobilization aid radio |
| | transmitter |
| 12 | Pedometer, comprising the transmitter 13, the acceleration sensor 14, the |
| | signal processing unit 15, the control unit 16 and the carrier 27 |
| 12.1 | Pedometer with reduced functionality, comprising the transmitter 13, the |
| | control unit 16 and the carrier 27 |
| 13 | Pedometer transmitter 12, 12.1 |
| 14 | Acceleration sensor of the pedometer 12 |
| 14.1 | Positioning unit, belongs to the computer system 110, measures the |
| | trajectory Tr.14 of the pedometer 12.1, uses signals from the receivers |
| | 40.1, 40.2, 40.3 |
| 15 | Signal processing unit of the pedometer 12, generates the signal Sig.12 |
| 16 | Control unit of the pedometer 12, 12.1, generates the signal Sig.12 |
| 17 | Wristband carrier 1, 1.1, on which the devices 2, 3, 4, 5, 6, 7, 8, 9 are |
| | mounted, can be worn on an arm or wrist, acts as a holding element |
| 18 | Local control unit of the wristband 1, 1.1 |
| 20 | Sensor for vital parameters, worn by the patient Pt on the right arm, |
| | includes the transmitter 22 |
| 21 | Company ID card of the caregiver Pk, includes a transmitter |
| 22 | Transmitter on the vital parameter sensor 20, emits a signal with the |
| | measured vital parameters |
| 27 | Carrier of the pedometer 12, acts as a holding element |
| 30 | Computer, comprises the output unit 34, the input unit 35, the |
| | communication unit 31, the comparison unit 32, and the database 33 |
| 31 | Communication unit of the computer 30, is in a data link with the |
| | communication unit 4 of the wristband 1, 1.1 |
| 32 | Comparison unit, implemented on the computer 30, compares the stored |
| | specifications Mü.1_{Spec}, Mü.2_{Spec}, Mü.3_{Spec} with the captured information |
| | Mü.1_{Info}, Mü.2_{Info}, Mü.3_{Info} |
| 33 | Database in which the mobilization plan Mp is stored and to which the |
| | comparison unit 32 has read access |
| 34 | Output unit of the computer 30 |
| 35 | Input unit of the computer 30 |
| 40.1, 40.2, 40.3 | Stationary receivers, receive signals from wristband 1.1 and pedometer |
| | 12.1 |
| 41 | Communication unit of the computer system 110 |
| 110 | Computer system, comprising the acceleration sensors 2.1 and 14.1, the |
| | determination unit 5.1, the signal processing unit 15.1, the computer 30 |
| | and the databases 9.1 and 33 |
| Bs.2 | Motion signal describing the movement of the wristband 1 generated by |
| | the acceleration sensor 2 |
| Bs.14 | Motion signal describing the movement of the pedometer 12 generated |
| | by the acceleration sensor 14 |
| dist.1 | Measured distance between the radio transmitter 10 on the cane St and |
| | the receiver 3 |
| dist.2 | Measured distance between the radio transmitter 11 on the wheelchair Rs |
| | and the receiver 3 |
| Mp | Mobilization plan, each includes a specification Mü.1_{Spec}, Mü.2_{Spec}, |
| | Mü.3_{Spec} for three mobilization exercises, is stored in the database 33 |
| Mü.1_{Info}, | Information captured on the actual performance of the three mobilization |
| Mü.2_{Info}, | exercises is compared by the comparison unit 32 with the specifications |
| Mü.3_{Info} | Mü.1_{Spec}, Mü.2_{Spec}, Mü.3_{Spec} |
| Mü.1_{Spec}, | Specifications for three mobilization exercises are part of the |
| Mü.2_{Spec}, | mobilization plan Mp |
| Mü.3_{Spec} | |
| ΔMü.1 | Deviation between the captured information Mü.1_{Info} and the stored |
| | specification Mü.1_{Spec}, determined by the comparison unit 32 |
| Pk | Caregiver who assists patient Pt in performing the mobilization exercises |
| | carries the company ID card 21 |
| Pt | Patient who is to perform mobilization exercises according to the |
| | mobilization plan Mp wears the wristband 1, 1.1, the pedometer 12, 12.1 |
| | and the vital parameters sensor 20 |
| Rs | Wheelchair, is the mobilization aid used in the second mobilization |
| | exercise, has the radio transmitter 11 |
| St | The mobilization aid, which is used in the first and third mobilization |
| | exercise, has the radio transmitter 10 |
| Tr.2 | Trajectory of the wristband 1.1, measured by the tracking unit 2.1 |
| Tr.14 | Trajectory of the pedometer 12.1, measured by the positioning unit 14.1 |

What is claimed is:

1. An arrangement for monitoring a mobilization plan for a human, the arrangement comprising:
a holding element;
a motion measuring device configured to measure a spatial movement of the holding element, wherein the motion measuring device comprises the holding element or is mechanically connected to the holding element or is or is configured to be data-link connected to the holding element and wherein the motion measuring device is configured to generate a motion signal, which signal describes the measured movement of the holding element;
a database configured to store a mobilization plan, wherein the database comprises the stored mobilization plan, which is stored in the database in a computer-evaluable form, and wherein the mobilization plan comprises a specification of a mobilization exercise, which exercise specifies a mobilization aid and a movement to be carried out with the aid of the mobilization aid, wherein the mobilization aid comprises a device which is configured to mechanically support a human in locomotion and/or to be moved by a human, wherein the holding element is configured to be worn on a human body of the human or to be attached to the mobilization aid;
a mobilization aid radio transmitter configured to be attached to the mobilization aid and configured to transmit a signal via radio waves, which signal comprises an identifier of the mobilization aid radio transmitter and/or an identification of the mobilization aid;
a reference element configured to be worn on the human body of the human;
a signal-processing verification unit, wherein the arrangement is configured to capture an identification which identifies the mobilization exercise specified in the stored mobilization plan, and wherein the verification unit is configured to check whether the mobilization aid specified in the captured mobilization exercise is actually used by a human wearing the reference element, wherein the verification unit is configured to measure at least one distance between the mobilization aid radio transmitter, which is attached to the mobilization aid, and the reference element, to use the signal from the mobilization aid radio transmitter for the distance measurement, and to use the measured distance for the check;
a signal-processing determination unit which is configured to determine information about a mobilization exercise actually performed and to use for this determination the motion signal and a result of the check by the verification unit, as to whether the mobilization aid specified in the identified mobilization exercise is actually used by a human wearing the reference element; and
a signal-processing comparison unit, which is configured to compare the determined information about the mobilization exercise actually performed with the specification of the mobilization exercise of the mobilization plan.

2. An arrangement according to claim 1,
wherein the verification unit is configured to measure a time course of the measured distance between the reference element and the mobilization aid radio transmitter and to use the signal from the mobilization aid radio transmitter to measure the time course of the measured distance, and
wherein the verification unit is further configured to use the measured distance time course for the check of whether the mobilization aid specified in the identified mobilization exercise is used by a human wearing the reference element.

3. An arrangement according to claim 1, further comprising a portable device adapted to be worn on the body of a human,
wherein the reference element comprises a receiver which is a component of the portable device, and
wherein the verification unit is configured to measure a distance between the mobilization aid radio transmitter and the receiver and to use the signal from the mobilization aid radio transmitter for the distance measurement.

4. An arrangement according to claim 3,
wherein the verification unit is another component of the portable device, and/or
wherein the determination unit is another component of the portable device.

5. An arrangement according to claim 3,
wherein the holding element is mechanically connected to or forms a part of the portable device adapted to be worn on the body of the human.

6. An arrangement according to claim 3, wherein the verification unit is configured as a stationary device and is configured to receive a signal from the portable device and to measure the distance between the mobilization aid radio transmitter and the receiver based on the signal from the portable device.

7. An arrangement according to claim 1,
wherein the mobilization exercise of the mobilization plan specifies a number of steps to be performed by a human with the mobilization aid specified in the mobilization exercise,
wherein the motion measuring device comprises a pedometer which is mechanically connected to the holding element or is data-link connected to the holding element,
wherein the pedometer is configured to count the steps performed by the human while the human is wearing the holding element, and
wherein the motion signal generated by the movement measuring device describes the measured number of steps.

8. An arrangement according to claim 1,
wherein the movement measuring device is configured to measure a trajectory of the holding element in a predetermined three-dimensional coordinate system, and
wherein the motion signal generated by the movement measuring device describes the measured trajectory.

9. A process for monitoring a mobilization plan for a human, the process comprising the steps of:
providing an arrangement, the arrangement comprising: a reference element; a holding element; a motion measuring device, and a mobilization aid radio transmitter, wherein the motion measuring device comprises the holding element or is mechanically connected to the holding element or is configured to be data-link connected to the holding element, wherein the reference element is worn at least temporarily by the human on his/her human body while the process is performed, wherein a mobilization plan is specified in a form that can be evaluated by computer, wherein the mobilization plan comprises a specification of a mobilization exercise, which exercise specifies a mobilization aid and a movement to be carried out with the aid of the mobilization aid, wherein the mobilization aid comprises a device which is configured to mechanically support the human in locomotion and/or to be moved by the human, wherein the holding element is worn on the human body by the human or is attached to the mobilization aid and the mobilization aid radio transmitter is attached to the mobilization aid while the process is performed;
with the mobilization aid radio transmitter, transmitting a signal by radio waves which signal comprises an identifier of the mobilization aid radio transmitter and/or of the connected mobilization aid;
capturing an identification of the mobilization exercise specified in the stored mobilization plan;
checking whether the mobilization aid specified in the captured mobilization exercise is actually used by the human wearing the reference element, wherein the checking comprises measuring a distance between the mobilization aid radio transmitter, which is attached to this mobilization aid, and the reference element, wherein the distance is measured based on the signal from the mobilization aid radio transmitter;
with the motion measuring device, measuring a movement of the holding element in space and generating a motion signal, which signal describes the measured movement of the holding element;
determining information about the mobilization exercise actually carried out by the human, the determination is based on the motion signal and the result of the checking whether the mobilization aid specified in the captured mobilization exercise is used by a human wearing the reference element, and
comparing the information determined about this mobilization exercise with the specification for the captured mobilization exercise of the mobilization plan.

10. A process according to claim 9,
wherein the mobilization plan additionally specifies a frequency and/or a duration with which the mobilization exercise is to be carried out,
wherein the step of determining information about the mobilization exercise determines a frequency and/or a duration of the mobilization exercise actually carried, and
wherein comparing the information determined about the mobilization exercise with the specification for the mobilization exercise in the mobilization plan further comprises comparing a determined frequency and/or duration of the mobilization exercise actually carried out with a specified frequency and/or duration in the mobilization plan for the mobilization exercise.
